(19) United States
(12) Patent Application Publication
(10) Pub. No.: US 2021/0244469 A1
(43) Pub. Date: Aug. 12, 2021

(54) MICROWAVE APPARATUS, SYSTEM AND METHOD

(71) Applicant: Emblation Limited, Alloa (GB)

(72) Inventors: Eamon McErlean, Alloa (GB); Gary Beale, Alloa (GB); Matthew Kidd, Alloa (GB); Shailesh Joshi, Alloa (GB)

(73) Assignee: Emblation Limited, Alloa (GB)

(21) Appl. No.: 17/273,554

(22) PCT Filed: Aug. 30, 2019
(86) PCT No.: PCT/GB2019/052438

§ 371 Date: Mar. 4, 2021

(30) Foreign Application Priority Data
Sep. 5, 2018 (GB) 1814399.0

(51) Int. Cl. A61B 18/18

(52) U.S. Cl. CPC A61B 18/1815; A61B 2018/1823; A61B 2018/1838; A61B 2018/1846

(57) ABSTRACT

A microwave system comprises: a microwave generator; and a microwave cable apparatus comprising a coaxial cable, wherein an exposed distal portion of an inner conductor of the coaxial cable is longer than an outer conductor of the coaxial cable, and wherein at least part of the exposed distal portion of the inner conductor is bent with respect to a longitudinal axis of the coaxial cable, thereby to provide a directional radiating element; wherein the microwave generator is configured to provide microwave energy to the cable apparatus at a frequency that provides directional radiation of microwave energy having a desired directionality from the radiating element.

### FIELD

The present disclosure relates to a microwave antenna apparatus or applicator for use in radiating microwave energy into tissues and onto tissue surface for medical applications. The present disclosure also relates to methods of fabricating a microwave antenna apparatus or applicator and to methods of radiating microwave energy into tissues and onto tissue surfaces.

### BACKGROUND

It is known to employ microwave applicators to radiate microwave energy into the tissues of a tumour for the purposes of increasing the temperature such that the tissues are ablated and the tumour is destroyed. Microwave ablation can also be used in healthy tissues such as when creating lines of conduction block by thermal damage rather than the incisions created in traditional Cox maze surgery for atrial fibrillation, where forming linear scars or lesions disrupt the transmission of the abnormal electrical impulses.

It is also known to employ microwave applicators to radiate microwave energy into the tissues of a diseased tissue, including pre-cancer, for the purposes of increasing the temperature so as to promote heating and activate biological effects within the tissue that may lead to disease irradiation and tissue repair. Such biological effects may occur at temperatures below the ablation threshold i.e. non-ablative.

Common to all microwave applicators for medical treatments is the use of an antenna to radiate microwave energy into tissue. A volume of tissue that is to be heated by microwave energy may be referred to as a target volume.

The antenna may be selected to be suitable for the tissue and disease type, location, geometry and power needs to achieve a therapeutic window. Typically, the antenna is electrically coupled to a microwave generator by a transmission line. The antenna is selected to generate repeatable and sufficient electromagnetic strength in a defined volume to provide reliable treatments.

Controlling the electrical field shape is important when seeking to achieve a dense or concentrated energy dose within tissues (for example, within a target volume of tissue). Protection of other adjacent tissues that may be sensitive to the thermal effects resultant in treated tissues must be considered in the design of an antenna. Adjacent tissues that are to be protected from microwave energy may be referred to as a protected volume. Tissues to be protected may comprise, for example, arteries, veins, nerves, and/or other important physiological features. The electromagnetic effects on nerves may require consideration to reduce the risk of damage. High electromagnetic fields may also be initiated inadvertently when metal is present either in the form of a tool or an implant and should be mitigated against. Directing the electric fields of an antenna is thus a common feature in microwave antenna for medical applications.

In the field of microwave tumour ablation, a common antenna design utilises the monopole principle where the centre conductor of a coaxial transmission line is exposed beyond the dielectric and shielding layers of the coaxial transmission line, continuing along its principal axis. The radial emission of electromagnetic field by the antenna is generally perpendicular to this principal axis and may offer predictable symmetrical heating of tissue. For optimal energy transmission into target tissues, the dimensions of the exposed conductor, adjoining dielectric materials and/or other features may be tuned to the operating frequency.

When seeking to shield the emission from a monopole antenna it is common practice to utilise the addition of a reflector coupled to the transmission line. The interaction of the antenna and reflector directs the electromagnetic field in an asymmetric manner when viewed along the principal axis of the antenna, compared to the unshielded monopole arrangement. The predetermined direction of bias provided by the antenna and reflector is then used to concentrate the field in the target volume and/or reduce or prevent electromagnetic fields in a protected volume. The emphasis on concentrating the field in the target volume and/or reducing or preventing electromagnetic fields in the protected volume may depend on user requirements for operation.

The reflector arrangement may vary in different systems. It may be offset but parallel to the principal axis or it may be at an angle, depending on the desired shape (Berube U.S. Pat. No. 6,245,062 B1). Further systems offset the centre conductor of the coaxial transmission line at the distal end of the antenna from the principal axis, reducing the gap from conductor to reflector (Berube U.S. Pat. No. 6,471,696 B1) and, as common to the requirements of optimal energy transmission into target tissues, require consideration of the same parameters as a standard monopole antenna.

A larger shape or 3D form may also be used (Shiu U.S. Pat. No. 8,672,933 B2) that extends substantially beyond the monopole antenna. A further known technique (Berube U.S. Pat. No. 6,245,062 B1, Gauthier U.S. Pat. No. 7,301,131 B2 and Prakash U.S. Pat. No. 8,690,869 B2) is to employ an electrically connected concentric tube or cradle featuring a window that allows the electromagnetic field from the contained monopole antenna to be emitted in that chosen section alone.

The use of a monopole antenna and reflector is common. Other antenna design types such as dipole, slot and helical can also be accommodated in designs.

The manufacturing methods for connecting a reflector component to an outer conductor may commonly use joining techniques such as soldering, brazing, ultrasonic welding or adhesive bonding. The manufacturing methods for connecting an antenna component to an inner conductor may commonly use joining techniques such as soldering, brazing, ultrasonic welding or adhesive bonding (Berube U.S. Pat. No. 6,245,062 B1).

### SUMMARY

In a first aspect of the invention, there is provided a microwave system comprising: a microwave generator; and a microwave cable apparatus comprising a coaxial cable, wherein an exposed distal portion of an inner conductor of the coaxial cable is longer than an outer conductor of the coaxial cable, and wherein at least part of the exposed distal portion of the inner conductor is bent with respect to a longitudinal axis of the coaxial cable, thereby to provide a directional radiating element. The microwave generator is configured to provide microwave energy to the cable apparatus at a frequency that provides directional radiation of microwave energy having a desired directionality from the radiating element.

The directional radiating element may provide a directional antenna that is made substantially from adaptions to the existing components of a coaxial transmission line with a minimal number of additional components, if any.

The addition of components to an antenna assembly may introduce losses at the interface between the transmission feed and the antenna. The losses introduced at the interface may reduce the overall efficiency of the antenna system. By forming the directional radiating element from a coaxial cable rather than adding further components, losses may be reduced.

The addition of components may increase complexity in the assembly that often translates to increased cost of manufacture. The complexity also introduces the risk of future component failure which is an especially important consideration in medical devices. By forming the directional radiating element from a coaxial cable rather than adding further components, complexity may be reduced.

The addition of components that use the window technique to achieve a directional antenna may impair the physical flexibility of the overall system that may be needed in catheter delivery systems or those that are space restricted.

The system may be configured to perform microwave ablation of tissue. The system may be configured to provide tissue hyperthermia. In use, directional radiation may be radiated into tissue from the directional radiating element to cause microwave ablation of tissue or tissue hyperthermia.

The exposed distal portion may be bent to form a hook shape.

The exposed distal portion may comprise a first part that is aligned with the principal axis of the coaxial cable and a second part that is substantially parallel with, and radially offset from, the first part.

The exposed distal portion may be bent with a bend angle of at least 90 degrees.

The exposed distal portion may be bent such that substantially all of the exposed distal portion lies within a radius of the coaxial cable.

The outer conductor of the coaxial cable may vary in length with respect to the circumference of the coaxial cable, thereby providing a shield element.

The system may further comprise a controller configured to select the frequency of the microwave energy provided to the cable apparatus and/or a power of the microwave energy provided to the cable apparatus.

The frequency and/or power may be selected in dependence on at least one of a reflection coefficient of the cable apparatus, a property of tissue to be treated, a volume of tissue to be treated, a type of treatment.

The desired directionality may comprise at least one of a desired depth of penetration into tissue, a desired radiation pattern, a desired linearity, a desired profile of radiated volume.

The coaxial cable may be flexible.

The system may further comprise a catheter or trocar into which the cable apparatus is insertable.

The cable apparatus may further comprise a dielectric cover configured to cover the radiating element. The dielectric cover may be biocompatible.

At least one of a plurality of design parameters may be selected in dependence on at least one of a volume of tissue to be treated, a property of tissue to be treated, a dielectric constant of tissue to be treated, a type of treatment. The design parameters may comprise at least one of: a cable dimension; a dimension of the radiating element; a length of the exposed distal portion of the inner conductor; a length of the radiating element; a bend radius of the radiating element; an offset distance between parts of the radiating element; a gap between the radiating element and the outer conductor; a size of a or the shield element; an arc of extent of a or the shield element; a shape of a or the shield element.

The frequency may be between 900 MHz and 30 GHz. The frequency may be about 915 MHz. The frequency may be about 2.45 GHz. The frequency may be about 5.8 GHz. The frequency may be about 8.0 GHz. The frequency may be about 24.125 GHz.

A diameter of the coaxial cable may be between 0.1 mm and 25 mm.

The radiating element may be alignable with a tissue feature, so as to radiate directionally into the tissue feature.

In a further aspect of the invention, there is provided a microwave cable apparatus comprising a coaxial cable, wherein an exposed distal portion of an inner conductor of the coaxial cable is longer than an outer conductor of the coaxial cable, and wherein at least part of the exposed distal portion of the inner conductor is bent with respect to a longitudinal axis of the coaxial cable, thereby to provide a directional radiating element.

In a further aspect of the invention, there is provided a method of fabricating a microwave cable apparatus. The method comprises: providing a coaxial cable, the coaxial cable comprising an inner conductor and an outer conductor; at a distal end of the coaxial cable, selectively removing a distal portion of the outer conductor to expose a distal portion of the inner conductor; and bending the exposed distal portion of the inner conductor to form a radiating element.

The directional microwave antenna is fabricated by selectively removing portions of the outer conductor and manipulating the centre conductor.

The removal of the outer conductor allows emission of the electromagnetic field in a specific region.

The manipulation of the distal portion of the central conductor of a monopole based design of antenna is used to create a longer resonating component and cause interactions with the centre conductor remaining with in the original dielectric material. The manipulation may comprise bending the centre conductor at the exit point of the inner dielectric through 180 degrees such that the conductor returns parallel to the principal axis of the transmission line.

With no additional components specifically needed to achieve directional and shielding performance, the cost and ease of manufacture may be advantageous over the current state of the art. Furthermore, with a reduced part count over directional antenna with additional reflector components the risk of failure at time of assembly or in later use may be reduced.

Improved efficiency of energy conversion may be achieved there is no interruption to the transmission line.

The directional antenna may have a compact size, The directional antenna may retain an inherent flexibility of the assembly that may be realised through the use of no extra component additions to reflect and shield the electromagnetic field. In particular, the compact size and/or flexibility may favour the demands of smaller or restrictive catheter based antenna delivered treatments.

The exposed distal portion may be bent to form a hook shape.

The exposed distal portion may comprise a first part that is aligned with the principal axis of the coaxial cable and a second part that is substantially parallel with, and radially offset from, the first part.

The exposed distal portion may be bent with a bend angle of at least 90 degrees.

The exposed distal portion may be bent such that substantially all of the exposed distal portion lies within a radius of the coaxial cable.

The method may further comprise tuning a resonance and/or a distribution of microwave energy to be emitted by the microwave cable apparatus by selecting at least one of: a cable dimension; a dimension of the radiating element; a length of the exposed distal portion of the inner conductor; a length of the radiating element; a bend radius of the radiating element; an offset distance between parts of the radiating element; a gap between the radiating element and the outer conductor.

The removing of the portion of the outer conductor may comprise selective removal of a portion of the outer conductor over a selected part of a circumference of the coaxial cable, thereby forming a shield element extending over a further part of the circumference of the coaxial cable.

The method may further comprise tuning a resonance and/or a distribution of microwave energy to be emitted by the microwave cable apparatus by selecting at least one of: a size of the shield element; an arc of extent of the shield element; a shape of the shield element.

The method may further comprise shaping a distal part of the shield element to form a hood.

In a further aspect of the invention, there is provided a method of performing a tissue heating process comprising: generating microwave energy by a microwave generator; providing the microwave energy to a microwave cable apparatus, the microwave cable apparatus comprising a coaxial cable, wherein an exposed distal portion of an inner conductor of the coaxial cable is longer than an outer conductor of the coaxial cable, and wherein at least part of the exposed distal portion of the inner conductor is bent with respect to a longitudinal axis of the coaxial cable, thereby to provide a directional radiating element; and heating tissue by directional radiation of microwave energy having a desired directionality from the radiating element.

The tissue heating may be so as to perform tissue ablation.

The tissue heating may be so as to cause tissue hyperthermia.

Features in one aspect may be applied as features in any other aspect, in any appropriate combination. For example, system features may be provided as method features or vice versa.

### BRIEF DESCRIPTION OF DRAWINGS

A microwave antenna apparatus, a microwave assembly and a microwave system will now be described by way of non-limiting example only with reference to the following figures of which:

FIG. 1 is a schematic illustration of a microwave system;

FIG. 2 is an illustration of a coaxial cable/transmission line construction/assembly;

FIG. 3A is an isometric illustration of a microwave antenna in accordance with an embodiment, where the microwave antenna is formed by selective outer conductor removal and manipulation of the centre conductor;

FIG. 3B is a cross section illustration of a microwave antenna in accordance with an embodiment, where the microwave antenna is formed by selective outer conductor removal and manipulation of the centre conductor;

FIG. 4A, FIG. 4B and FIG. 4C are side elevation representations of the distribution of electromagnetic fields and microwave fields in particular, in the form of SAR (surface absorption rate) for 3 microwave antennae formed by selective outer conductor removal and manipulation of the centre conductor with varying lengths of emitting region;

FIG. 5A, FIG. 5B and FIG. 5C are planar representations of the distribution of electromagnetic fields and microwave fields in particular, in the form of SAR (surface absorption rate) for 3 microwave antennae formed by selective outer conductor removal and manipulation of the centre conductor with varying lengths of emitting region;

FIG. 6 illustrates the scattering parameter S11, a mathematical construct quantifying how RF energy propagates through the microwave antenna apparatus into the tissue when varying lengths of emitting region;

FIG. 7A, FIG. 7B and FIG. 7C are side elevations representation of the distribution of electromagnetic fields and microwave fields in particular, in the form of SAR (surface absorption rate) for 3 microwave antennae formed by selective outer conductor removal and manipulation of the centre conductor with varying gaps between centre conductor and outer conductor;

FIG. 8A, FIG. 8B and FIG. 8C are planar representations of the distribution of electromagnetic fields and microwave fields in particular, in the form of SAR (surface absorption rate) for 3 microwave antennae formed by selective outer conductor removal and manipulation of the centre conductor with varying gaps between centre conductor and outer conductor;

FIG. 9 illustrates the scattering parameter S11, a mathematical construct quantifying how RF energy propagates through the microwave antenna apparatus into the tissue when varying gaps between centre conductor and outer conductor;

FIG. 10A, FIG. 10B and FIG. 10C are side elevations representation of the distribution of electromagnetic fields and microwave fields in particular, in the form of SAR (surface absorption rate) for 3 microwave antennae formed by selective outer conductor removal and manipulation of the centre conductor with varying offset between centre conductor principal axis and the return/bent axis;

FIG. 11A, FIG. 11B and FIG. 11C are planar representations of the distribution of electromagnetic fields and microwave fields in particular, in the form of SAR (surface absorption rate) for 3 microwave antennae formed by selective outer conductor removal and manipulation of the centre conductor with varying offset between centre conductor principal axis and the return/bent axis;

FIG. 12 illustrates the scattering parameter S11, a mathematical construct quantifying how RF energy propagates through the microwave antenna apparatus into the tissue when varying offset between centre conductor principal axis and the return/bent axis;

FIG. 13A, FIG. 13B and FIG. 13C are side elevations representation of the distribution of electromagnetic fields and microwave fields in particular, in the form of SAR (surface absorption rate) for 3 microwave antennae formed by selective outer conductor removal and manipulation of the centre conductor with varying arc of extent at the distal tip;

FIG. 14A, FIG. 14B and FIG. 14C are planar representations of the distribution of electromagnetic fields and microwave fields in particular, in the form of SAR (surface absorption rate) for 3 microwave antennae formed by selective outer conductor removal and manipulation of the centre conductor with varying extent of the outer conductor removal at the distal tip;

FIG. 15 illustrates the scattering parameter S11, a mathematical construct quantifying how RF energy propagates through the microwave antenna apparatus into the tissue when varying extent of the outer conductor removal at the distal tip;

FIG. 16A, FIG. 16B, FIG. 16C and FIG. 16D are side elevations representation of the distribution of electromagnetic fields and microwave fields in particular, in the form of SAR (surface absorption rate) for 3 microwave antennae formed by selective outer conductor removal and manipulation of the centre conductor with varying outer conductor removal form;

FIG. 17A, FIG. 17B, FIG. 17C and FIG. 17D are planar representations of the distribution of electromagnetic fields and microwave fields in particular, in the form of SAR (surface absorption rate) for 3 microwave antennae formed by selective outer conductor removal and manipulation of the centre conductor with varying outer conductor removal form;

FIG. 18 illustrates the scattering parameter S11, a mathematical construct quantifying how RF energy propagates through the microwave antenna apparatus into the tissue when varying outer conductor removal form; and

FIG. 19. and FIG. 20 are photographic images of ex-vivo bovine liver that is heated using the directional antenna of an embodiment.

### DETAILED DESCRIPTION OF THE DRAWINGS

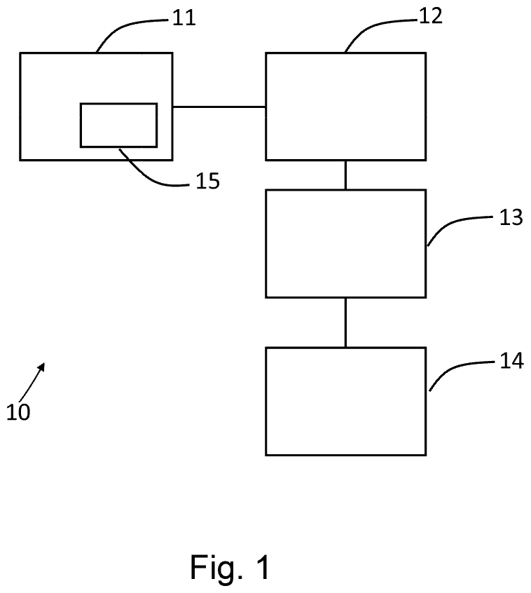

FIG. 1 illustrates a microwave system generally designated 10 for treating a tissue. The microwave system 10 comprises a microwave generator 11 for providing microwave energy, a flexible interconnecting microwave cable such as a coaxial cable 12, a hand grip or hand piece 13, and a microwave antenna apparatus 14. The microwave generator 11 comprises a controller 15 configured to select a frequency of microwave energy provided to the cable apparatus and/or a power of microwave energy provided to the cable apparatus.

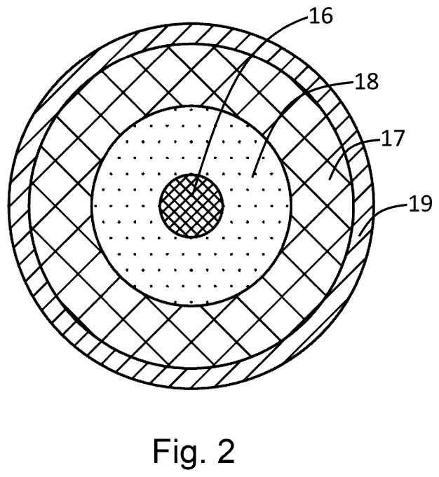

FIG. 2 is a cross-sectional illustration of a coaxial cable that may be used as a flexible interconnecting microwave cable 12 in the system of FIG. 1. The coaxial cable may also be referred to a transmission line. The construction of a typical transmission line (coax) shown in FIG. 2 includes a flexible coaxial transmission line (coax) including a flexible centre conductor 16 coaxial with a flexible cylindrical outer conductor 17. An insulating material 18 substantially fills the space between centre conductor 16 and outer conductor 17. The insulating material 18 may also be referred to as a dielectric material. The insulating material 18 is for holding the centre conductor 16 and outer conductor 17 in place and for electrically isolating the conductors from each other.

The outer conductor 17 may be referred to as a primary outer conductor. The primary outer conductor 17 may be augmented with a second flexible conductive sheath or braid (not shown), which may be positioned outside the primary outer conductor 17.

In turn, the outer conductor 17 or the second flexible conductive sheath or braid may be coated over its length by a flexible jacket 19. The flexible jacket 19 may be made of an inert impermeable and low friction material, for example FEP (Fluorinated ethylene propylene). A suitable type of coaxial transmission line is manufactured by HUBER+SUHNER (Switzerland) reference by type SUCOFORM_43_FEP_MED having nominal outer FEP jacket diameter of 1.09 mm, a dielectric diameter of 0.84 and flexible centre conductor diameter of 0.31 mm.

In other embodiments, other coaxial transmission lines may be used, for example coaxial transmission lines having different dimensions and/or formed from different materials. In some embodiments, the coaxial cable may be semi-rigid or rigid.

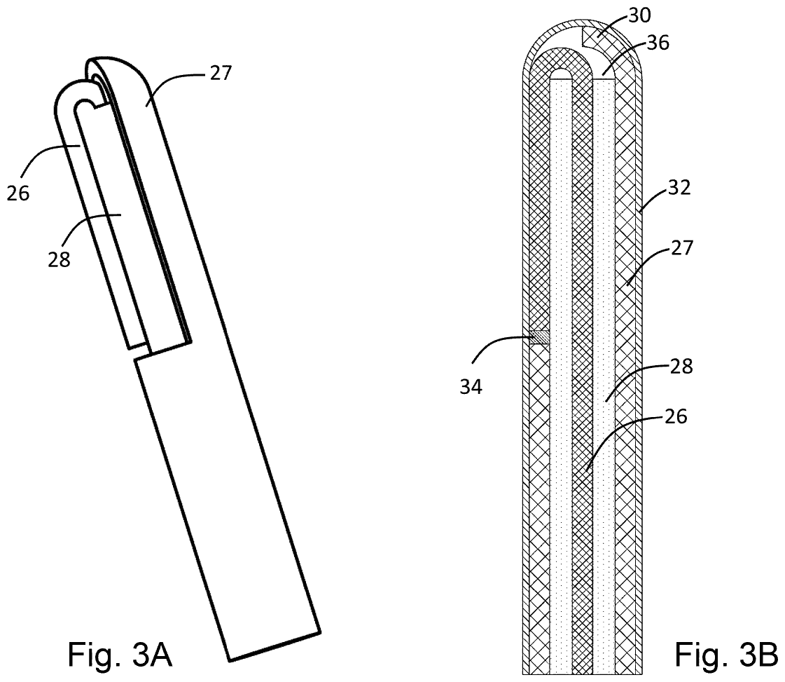

FIG. 3A and FIG. 3B are schematic illustrations of an antenna according to an embodiment. The antenna is formed from a coaxial cable (for example, a coaxial cable as illustrated in FIG. 2). FIG. 3A is an isometric view. FIG. 3B is a cross-sectional view. The coaxial cable comprises an inner conductor 26, an outer conductor 27, an inner dielectric 28 between the inner conductor 26 and outer conductor 27, and a flexible jacket around the outer conductor (the flexible jacket is not shown in FIG. 3A and FIG. 3B). In other embodiments, the coaxial cable may comprise additional components, for example an additional sheath or braid around the outer conductor 27.

The manufacture of the formed antenna in FIG. 3 is made in a series of steps. First, a first portion of the flexible jacket, the outer conductor 27 and the inner dielectric 28 over a first predetermined length is removed to expose a first portion of the centre conductor 26. This first removal of cable components comprises removing the flexible jacket, outer conductor 27 and inner dielectric 28 over a full 360 degrees around the cable. In the present embodiment, the first portion of the flexible jacket, outer conductor 27 and inner dielectric 28 is removed by cutting. The cutting of these materials may be carried out by knife, laser or other means that leaves the underlying dielectric 28 undamaged and permits the removal of the segments without difficulty. After the first step, the first portion of the centre conductor 26 protrudes from the rest of the coaxial cable. The corresponding first portion of the flexible jacket, outer conductor 27 and inner dielectric 28 has been removed.

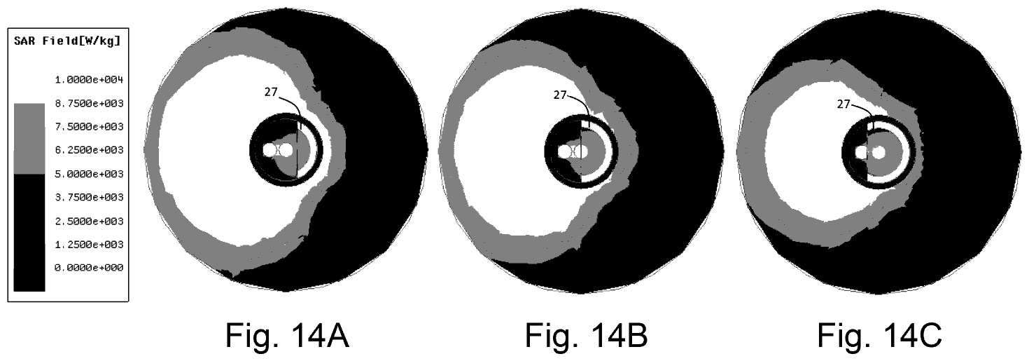

A second step comprises a segment wise removal of a second portion of the flexible jacket, outer conductor 27 and inner dielectric 28 having a second predetermined length. In the segment wise removal, a segment of the flexible jacket, outer conductor 27 and inner dielectric 28 is removed. The segment extends longitudinally over the whole second portion, but includes only a part of the circumference of the coaxial cable. Examples of segment shapes are discussed below with reference to FIGS. 14A to 14B. Again, cutting of the flexible jacket, outer conductor 27 and inner dielectric 28 may be carried out by knife, laser or other means that leaves the underlying dielectric 28 undamaged.

In a third step, the fully-exposed first portion of the centre conductor is bent through 180 degrees at the point where it is first exposed by the inner dielectric 28. The centre conductor is bent back on itself to form a hook shape. A directional radiating element is formed by the bending of the centre conductor. The formability of the centre conductor is dependent on the material properties. Solid copper commonly used in such transmission lines, is also readably permanently formable and retains the bent shape.

Various parameters of the radiating element may be chosen in order to provide a desired resonance frequency and/or a desired directionality of radiation emitted by the radiating element. For example, a length of the element may be selected. A bend radius of the element may be selected. A gap between the element and the outer conductor may be selected. Selection of these parameters is discussed in detail below with reference to simulations.

In the simplest form the antenna after the third step may be suitable for use. Other optional steps may improve performance and suitability with certain tissues.

In the embodiment shown in FIGS. 3A and 3B, the top of the outer conductor 27 is deformed to form a hood 30. The outer conductor 27 may be considered to comprise a two-dimensional sheet. The hood 30, which may also be referred to as a cap, is a three-dimensional shape which is formed from the outer conductor 27. Portions of the top of the outer conductor may be overlapped to form the hood. Portions of the top of the outer conductor may be stitched together to form the hood.

An optional manufacturing step may include the addition of dielectric material to the outer surface of the antenna to reduce the risk of short circuiting the antenna though a highly conductive tissue. Suitable materials for the dielectric covering may include, for example, FEP, PTFE, Silicone rubber, Polyolefin, Elastomer, Polyvinyl and Fluoropolymer. A dielectric covering 32 is shown in FIG. 3B.

An optional manufacturing step may include the addition of a dielectric material component located in the gap between the bend segment of the centre conductor 26 and the distal portion 30 of the outer conductor. Such a dielectric material component 36 is shown in FIG. 3B. Suitable materials for the dielectric component may include, for example, FEP, PTFE, Silicone rubber, Polyolefin, Elastomer, Polyvinyl and Fluoropolymer. In further embodiments, the component 36 may be formed from the inner dielectric component 28 at the time of step 1 where a portion of the inner dielectric 28 is left in place.

An optional manufacturing step may include the addition of a dielectric material component located in the gap between the cut end of the centre conductor 26 (after it has been bent down) and the part of the cut outer conductor 27 that the centre conductor 26 has been bent down towards. This component 34 shown in FIG. 3B may prevent a short between the centre and inner conductors 26, 27. Suitable materials for the dielectric component may include, for example, FEP, PTFE, Silicone rubber, Polyolefin, Elastomer, Polyvinyl and Fluoropolymer.

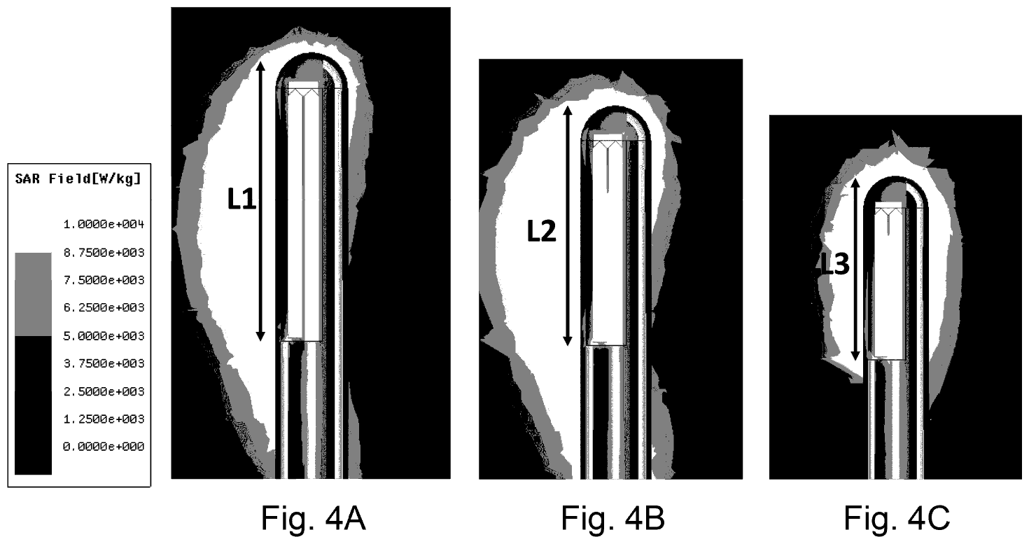

An optional manufacture step may occur before step 3, before the inner conductor is formed by bending. When the distance between the virgin inner conductor and the formed centre conductor is desired to be less than the radius of the inner conductor, a groove or slot may be formed in the inner dielectric that sets the desired distance and permits the forming of the centre conductor into said groove or slot. The application of the antenna and microwave system are considered in the antenna design. The embodiments shown in FIGS. 4 to 18 relate to the modelling of antenna designs in a tissue of relative permittivity (Er) of 40 and loss tangent (tanσ) of 0.5, representing an example tissue similar to a human cervix. The operating frequency of 8 GHz is used as the microwave generator. Other embodiments may be suitable for tissues in other ranges of Er, for example ranges within an overall Er range of 1 to 100, which may be representative of tissue. Other embodiments may use a microwave generator operating at any suitable frequency, for example 915 MHz or 2.54 GHz to 15 GHz.

In use, the microwave generator 11 generates microwave energy. The microwave generator 11 supplies microwave energy to the coaxial probe 12, and at least some of the supplied microwave energy is radiated from the microwave antenna apparatus 14. The microwave antenna apparatus 14 is placed near to or contacting the tissue of a patient. Microwave energy is radiated by the end surface 12 into the tissue of the patient.

The microwave generator is configured to provide microwave energy to the microwave antenna apparatus 14 at a frequency that provides directional radiation from the microwave antenna apparatus 14. The frequency may be selected such that the directional radiation has a desired directionality, for example a desired radiation pattern.

The directional antenna of FIGS. 3A and 3B is made substantially from adaptions to the existing components of a coaxial transmission line with a minimal number of additional components, if any. The addition of components to an antenna assembly may introduces losses at the interface between the transmission feed and the antenna. The losses introduced at the interface may reduce the overall efficiency of the antenna system.

The addition of components may increase complexity in the assembly that may often translate to increased cost of manufacture. The complexity may also introduce the risk of future component failure which may be an especially important consideration in medical devices.

The addition of components that use the window technique to achieve a directional antenna may impair the physical flexibility of the overall system that may be needed in catheter delivery systems or those that are space restricted. In contrast, the antenna of FIGS. 3A and 3B may be physically flexible.

In some circumstances, the selective removal of the outer conductor may allow emission of the electromagnetic field in a specific region in a similar manner as may be achieved using a window method in which a section of a secondary component removed to allow emission in a region.

In the antenna of FIGS. 3A and 3B, manipulation of the distal portion of the central conductor of a monopole based design of antenna is used to create a longer resonating component and cause interactions with the centre conductor remaining with in the original dielectric material. The manipulation comprises bending the centre conductor at the exit point of the inner dielectric through 180 degrees such that the conductor returns parallel to the principal axis of the transmission line.

With no additional components specifically needed to achieve directional and shielding performance, the cost and ease of manufacture may be advantageous over existing antennas. Furthermore, with a reduced part count over directional antenna with additional shield components the risk of failure at time of assembly or in later use may be reduced.

Improved efficiency of energy conversion may be achieved as there is no interruption to the transmission line.

The antenna has a compact size. The antenna may retain an inherent flexibility of the cable assembly that is realised through the use of no extra component additions needed to reflect and shield the electromagnetic field. In particular, this aspect may favour the demands of smaller or restrictive catheter based antenna delivered treatments.

The embodiments discussed below with reference to FIGS. 4 to 18 have been simulated using a 3D simulation model. In this case, the simulation model is HFSS (Ansoft Corp) which is a Finite Element Method (FEM) based full wave electromagnetic solver. Any appropriate simulation method may be used to simulate antennas according to embodiments.

Simulations may allow the calculation of a predicted response for coupling efficiency and specific absorption rate (SAR). SAR is a measure of the rate at which energy is absorbed by the human body when exposed to a radio frequency (RF) electromagnetic field. Simulations may also allow the calculation of how much power is reflected back from the antenna, a common reference parameter in regards to antennas, known as S₁₁,. The parameter of return loss, RL of an antenna system also a common term and is calculated thus RL=−S₁₁. A low return loss signifies a well matched (high efficiency) antenna.

It has been found that an impact on antenna performance may result when changing the emitting region dimensions. Changing dimensions of an emitting region may be of use when designing for a specific tissue, energy field profile and frequency combination.

Three embodiments, each with a different emitting length L1, L2, L3 are shown in FIGS. 4A to 4C. The emitting length L1, L2, L3 is the length along the principal axis of the coaxial cable from a point at which the inner conductor 27 has been at least partially exposed to the tip of the antenna. In the embodiments of FIGS. 4A to 4C, the tip of the antenna is the top of the hood 30 that is formed by shaping the cut end of the outer conductor 17. The antenna of FIG. 4A has the longest emitting length L1; the antenna of FIG. 4B has an intermediate emitting length L2; and the antenna of FIG. 4C has the shortest emitting length L3.

In FIGS. 4A to 4C, simulated SAR is shown in cross section. SAR for each antenna is shown to be different. The direction bias is more prominent (the antenna is more directional) with a longer emitting region L1, if all the other parameters are kept the same.

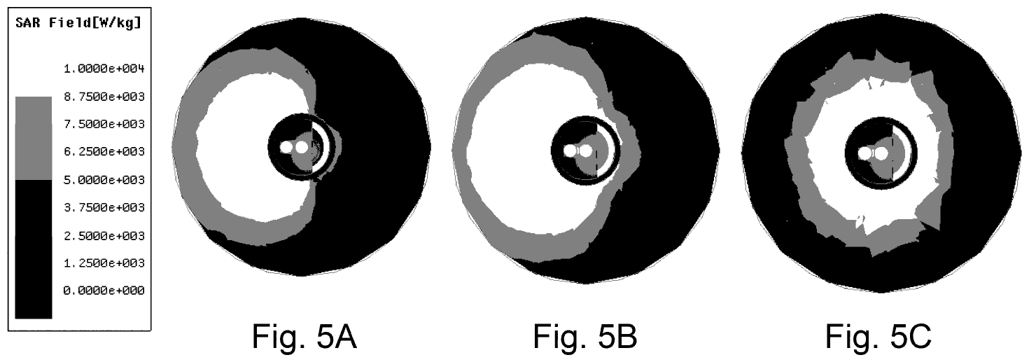

FIGS. 5A, 5B and 5C provide a planar view of the antennas shown in FIGS. 4A, 4B and 4C respectively and their simulated SAR. The planer view of SAR shown in FIG. 5 also shows that each length configuration L1, L2, L3 concentrates the direction bias to a different degree, if all the other parameters are kept the same.

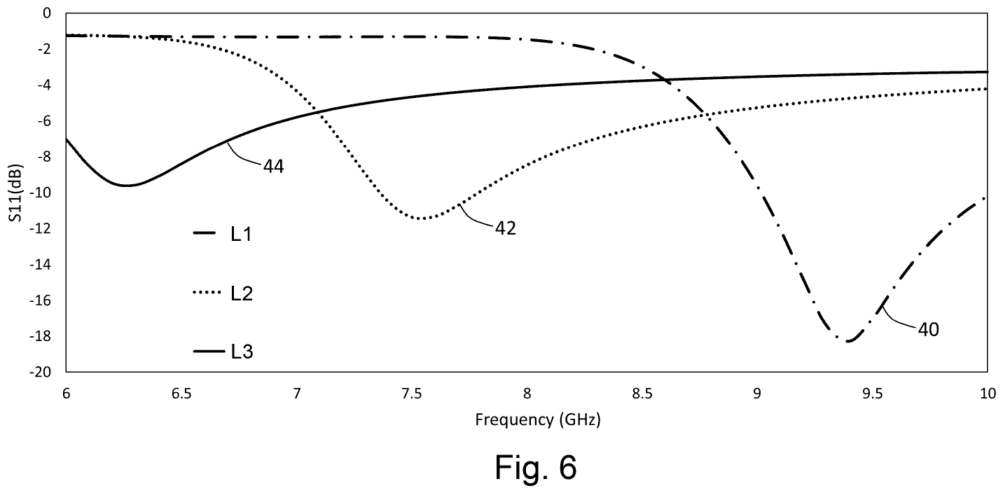

FIG. 6 plots the simulated S11 for the antennas shown in FIG. 4A (line 40), FIG. 4B (line 42) and FIG. 4C (line 44). The return loss is also affected by changes to the emitting length with longer lengths more resonant (dip in S₁₁) at a higher frequency compared to shorter lengths resonating at a lower frequency.

It has been found that an impact on antenna performance may result when changing a gap dimension between the distal end of the centre conductor 26 and the outer conductor 27. The gap is a distance between a cut end of the centre conductor 26 (after it has been bent down) and the cut end of the outer conductor 27. The size of the gap may be changed by changing the relative lengths of the first portion of the coaxial cable that is removed and the second segment of the coaxial cable that is removed.

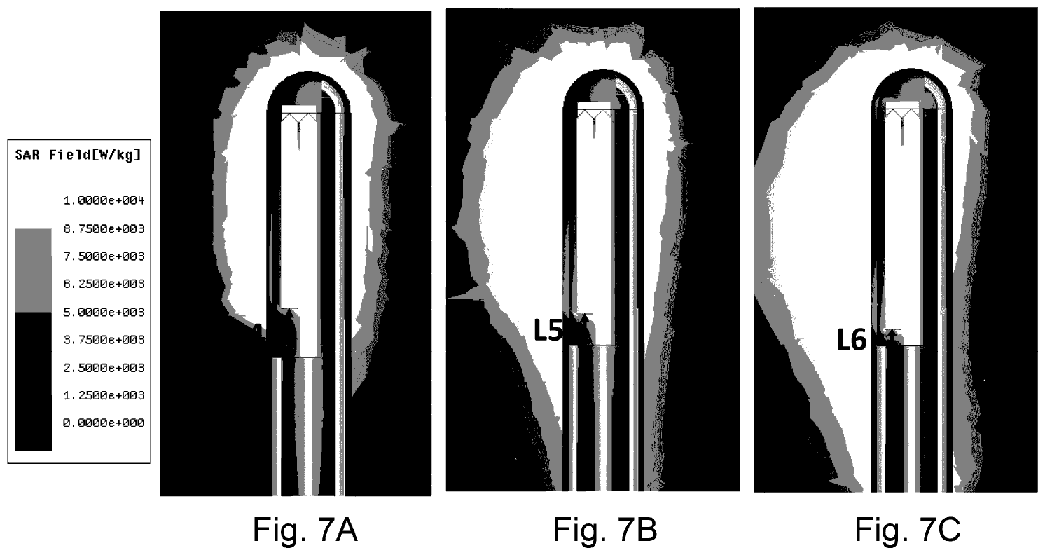

Changing the gap dimension may be of use when designing for a specific tissue, energy field profile and frequency combination. Three embodiments, each with a different gap dimension are shown in FIG. 7 the simulated SAR for the antennas shown in FIGS. 7A to 7C respectively. The SAR is shown in cross section. SAR for each antenna is shown to be different. The direction bias is more prominent with a shorter gap dimension, if all the other parameters are kept the same.

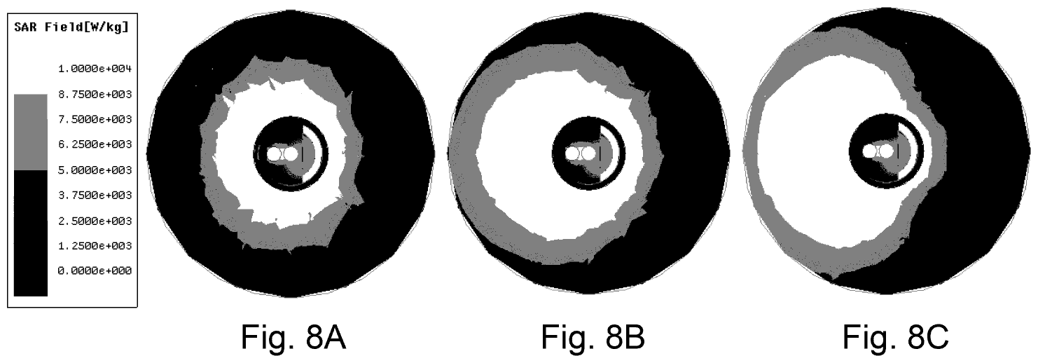

FIGS. 8A, 8B and 8C provide a planar view of the antennas shown in FIGS. 7A, 7B and 7C respectively and their simulated SAR. The planar view of SAR shown in FIGS. 8A to 8C also shows each gap dimension configuration to concentrate the bias to a different degree, if all the other parameters are kept the same.

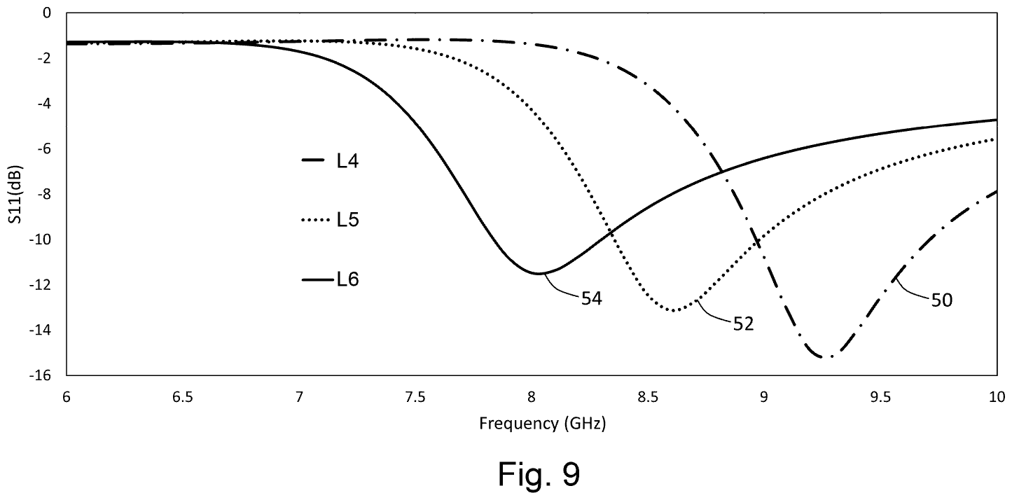

FIG. 9 plots the simulated S11 for the antenna shown in FIG. 7A (line 50), the antenna shown in FIG. 7B (line 52), and the antenna shown in FIG. 7C (line 54). The return loss is also affected by changes to the gap dimension; with a smaller gap more resonant (dip in S₁₁) at a higher frequency compared to a larger gap resonating at a lower frequency.

It has been found that an impact on antenna performance may result when changing an offset dimension between the centre conductor principal axis (which is the principal axis of the original coaxial cable) and the return/bent axis (which is the longitudinal axis of the part of the centre conductor that runs parallel to the original centre conductor axis after the centre conductor has been bent by 180 degrees). Changing the offset dimension may be of use when designing for a specific tissue, energy field profile and frequency combination.

Three embodiments, each with a different offset dimension are shown in

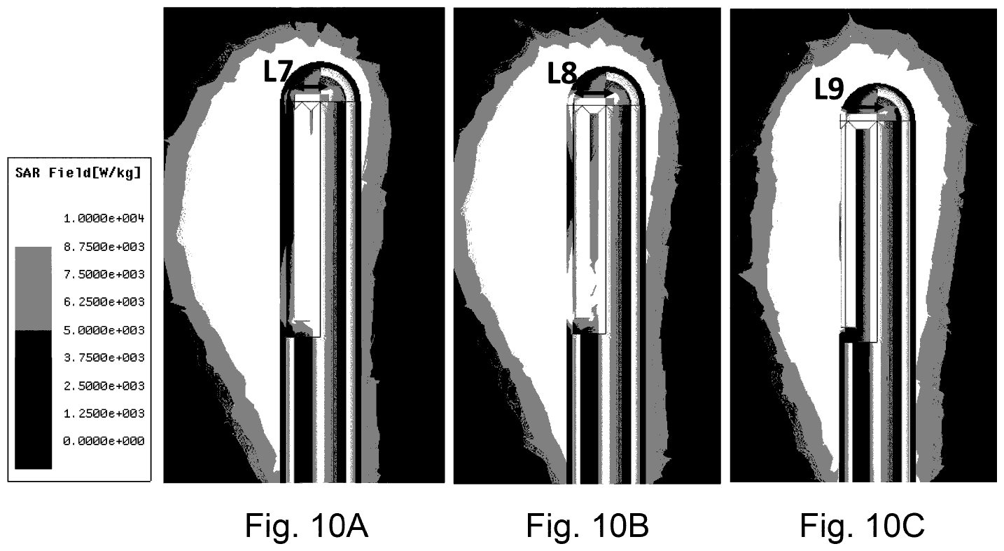

FIGS. 10A to 10C. The antenna of FIG. 10A has the smallest offset dimension L7. The antenna of FIG. 10B has an intermediate offset dimension L8. The antenna of FIG. 10C has the largest offset dimension L9. The SAR is shown in cross section. The SAR for each offset dimension is shown to be different. The direction bias is more prominent with a shorter offset dimension, if all the other parameters are kept the same.

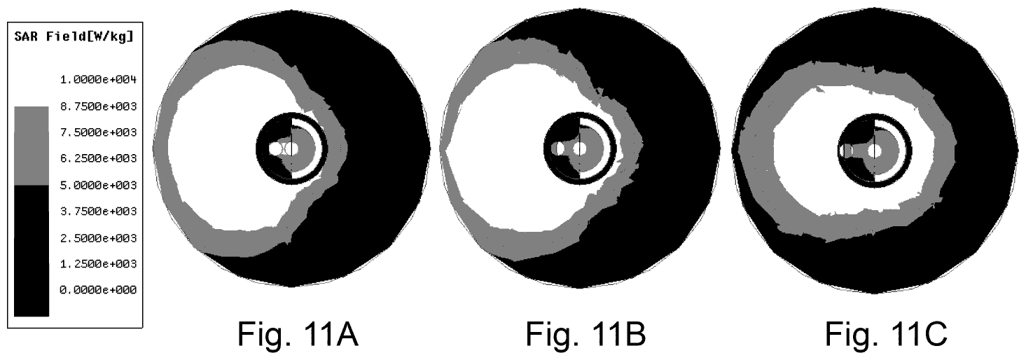

FIGS. 11A, 11B and 11C provide a planar view of the antennas shown in FIGS. 10A, 10B and 10C respectively and their simulated SAR. The planar view of SAR shown in FIGS. 11A to 11C also shows each offset dimension configuration to concentrate the bias to a different degree, if all the other parameters are kept the same.

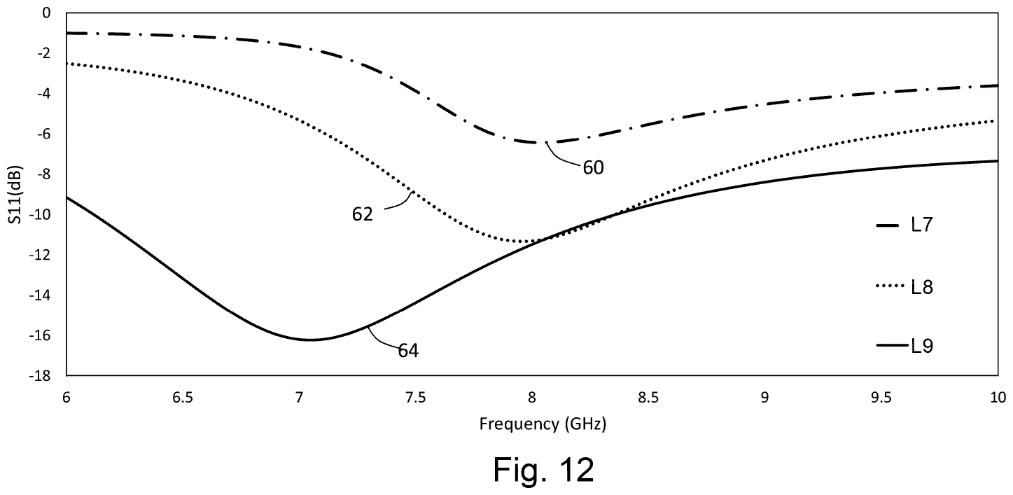

FIG. 12 plots the simulated S11 for the antenna shown in FIG. 10A (line 60), the antenna shown in FIG. 10B (line 62), and the antenna shown in FIG. 10C (line 64).

The return loss is also affected by changes to the offset dimension, with a smaller offset dimension resonant (dip in S₁₁) at a higher frequency compared to a larger offset dimension resonating at a lower frequency.

It has been found that impact on antenna performance may result when changing the arc of extent of the outer conductor 27 at the distal end. Changing the arc of extent may be of use when designing for a specific tissue, energy field profile and frequency combination. The arc of extent relates to the size of the part of the outer conductor 27 remains after step 2 of the forming process. As described above, only a segment of the outer conductor 27 is removed at step 2. The arc of outer conductor that remains after step 2 may be described in terms of arc of extent.

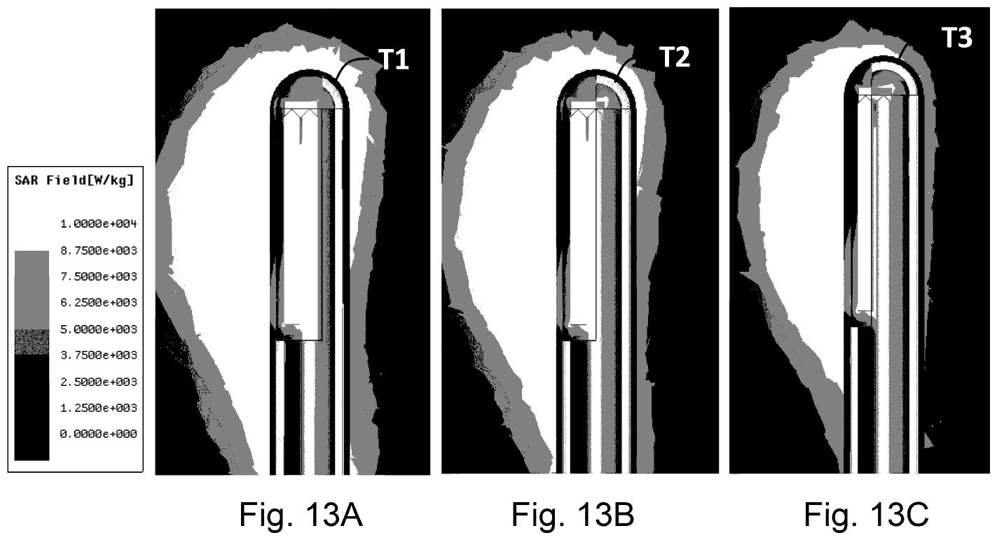

Three embodiments of antennas, each with a different arc of extent in cross section dimension are shown in FIG. 13A (smallest arc of extent), FIG. 13B (intermediate arc of extent) and FIG. 13C (largest arc of extent). In FIG. 13A, the arc of extent is 60 degrees. In FIG. 13B, the arc of extent is 90 degrees. In FIG. 13C, the arc of extent is 120 degrees.

The arcs of extent may also be seen clearly in the planar plots of FIGS. 14A to 14C, which show the simulated SAR for the antennas of FIGS. 13A to 13C respectively. The part of the outer conductor 27 retained after removal of the segment at step 2 is shown in the SAR plots.

The different arcs of extent result in different sizes of hood, where the hood is formed from the distal tip of the outer conductor 27. The hood in the antenna of FIG. 13C is larger than the hood in the antenna of FIG. 13A.

In FIGS. 13A to 13C, the simulated SAR is shown in cross section. SAR for each antenna is shown to be different. The direction bias is more prominent with a larger arc extent dimension, if all the other parameters are kept the same.

FIGS. 14A, 14B and 14C provide a planar view of the antennas shown in FIGS. 13A, 13B and 13C respectively and their simulated SAR. The planar view of SAR shown in FIGS. 14A to 14C also shows each arc extent dimension configuration to concentrate the bias to a different degree, if all the other parameters are kept the same.

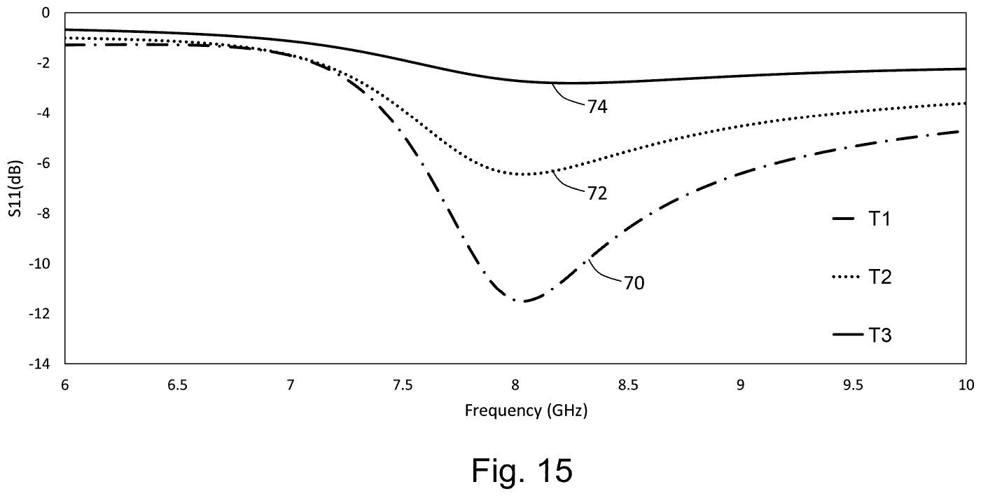

FIG. 15 plots the simulated S11 for the antenna shown in FIG. 10A (line 70), the antenna shown in FIG. 10B (line 72), and the antenna shown in FIG. 10C (line 74).

The return loss is also affected by changes to the arc extent dimension, with a smaller arc dimension more resonant (magnitude of dip in S₁₁) at the same frequency compared to a larger are dimension resonating less.

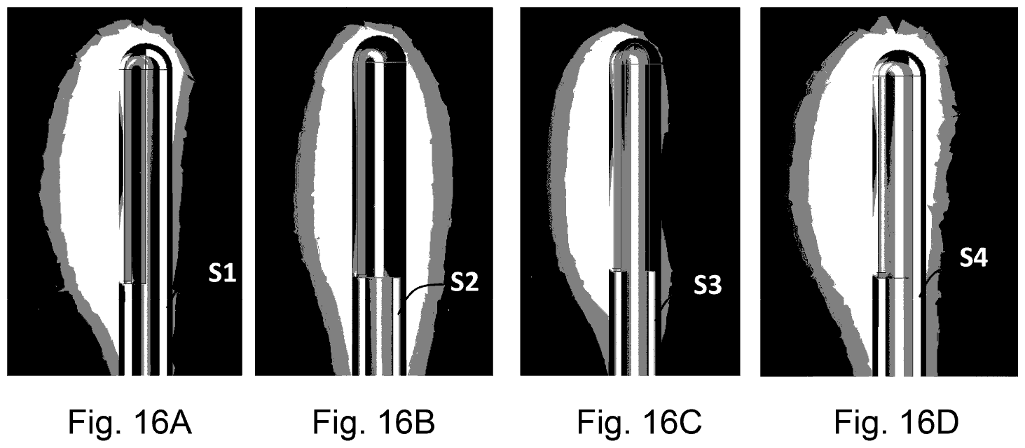

It has been found that an impact on antenna performance may result when changing the dimensional form of the outer conductor removal. Changing the form of the part of the outer conductor 27 that is removed may be of use when designing for a specific tissue, energy field profile and frequency combination. Four embodiments, each with a different dimension form of outer conductor removal are shown in FIGS. 16A to 16D.

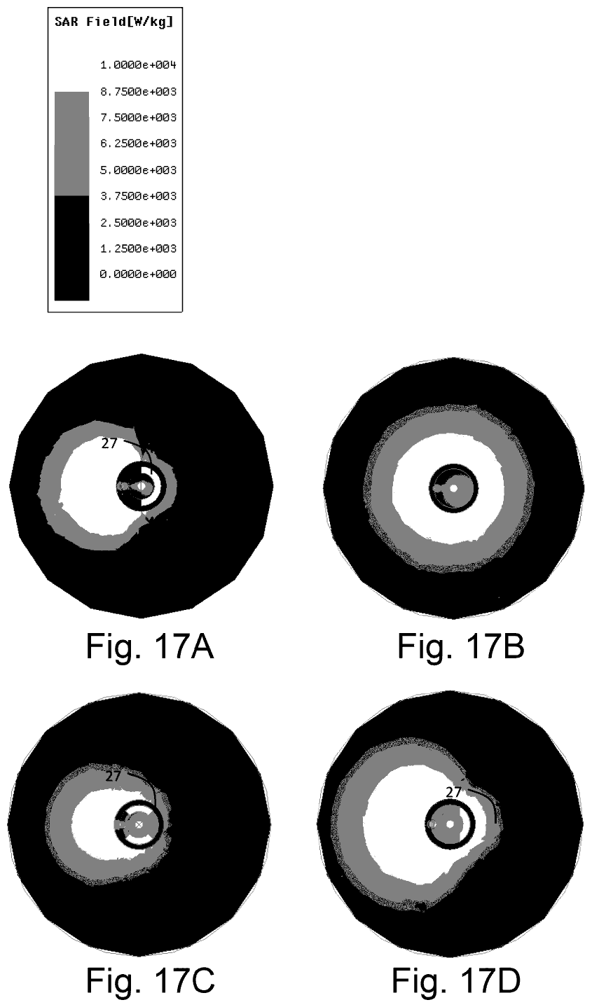

The embodiment of FIG. 16A comprises a forming of the outer conductor 27 for the length of the antenna section with a 180 degree arc of extent in axial cross section, as shown in the associated cross section FIG. 17A. In the embodiment of FIG. 16B, the outer conductor is removed entirely for the full length of the radiating element. In the embodiment of FIG. 16C, a segment of outer conductor at the front of the antenna and a segment of outer conductor at the back of the antenna are both removed. In the embodiment of FIG. 16D, an outer conductor with a very small arc of extent is used. In embodiments where portions or segments of the outer conductor are removed the gap left can be left as air or filled with a dielectric component. Suitable materials may include, for example, FEP, PTFE, Silicone rubber, Polyolefin, Elastomer, Polyvinyl and Fluoropolymer.

It was found that all of the antennas of 16A to 16D radiated directionally. Directional radiation was obtained even when there was no reflector present, as shown in FIG. 16B.

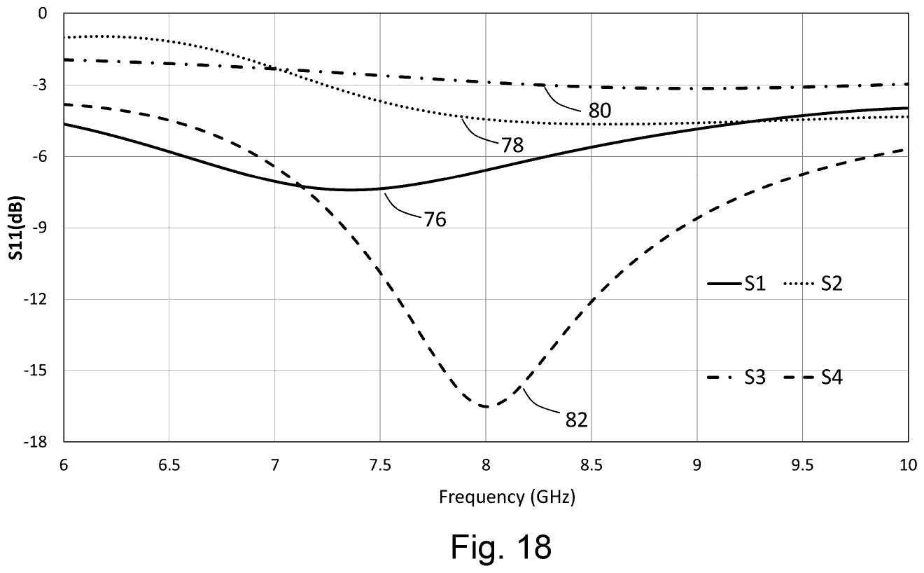

In FIGS. 16A to 16D, the SAR is shown in cross section. SAR for each antenna is shown to be different. The direction bias shape is different for different dimensional forms, if all the other parameters are kept the same. In FIG. 16A where the arc of extent is 180 degrees there is a pronounced bias and shielding effect. In FIG. 16B where there is no outer conductor present for the antenna section, there is a near uniform radiation and no shielding element. In FIG. 16C the segmentation of the outer conductor creates a more focussed bias than FIG. 16A and maintains high shielding effect. In FIG. 16D there is a similar radiation bias to FIG. 16A but a lesser shielding effect. The planar view of SAR shown in FIG. 17 also shows each dimensional form configuration concentrates the bias to a different pattern, with all the other parameters the same. The return loss (S₁₁) is also affected by changes to the dimensional form; these effects are shown in FIG. 18.

A specific antenna embodiment may be formed from a combination of any of design features disclosed above. The design features, which may also be referred to as design parameters, may include, for example:

cable dimensions;

emitting region dimensions;

dimension between the distal end of the centre conductor and the outer conductor;

offset dimension between centre conductor principal axis and the return/bent axis;

arc of extent of the outer conductor at the distal end;

and dimensional form of the outer conductor removal.

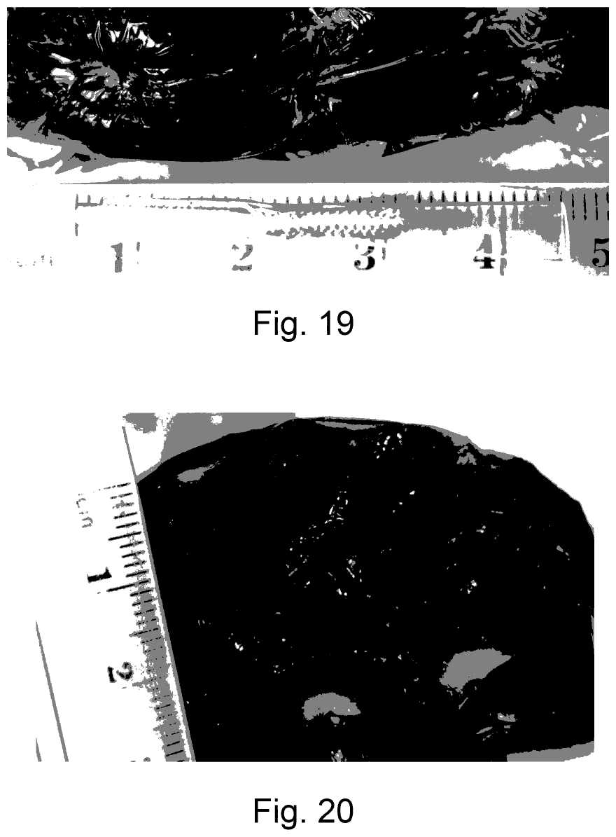

An example embodiment was evaluated with ex-vivo bovine liver. Images of the ex-vivo bovine liver are shown in FIG.19 and FIG. 20. Liver is a common reference material for medical microwave antenna testing as the tissue changes colour to a blanched region that is fixed post energising and allows the affected zone to be measured and compared against computer simulations or other antenna designs. The cable used was manufactured by HUBER+SUHNER (Switzerland) referenced by type MULTIFLEX_53, the overall emitting dimension was 7.35 mm, the gap dimension was 1.2, arc of extent of the outer conductor at the distal end was 90 degrees and the dimensional form of outer conductor matched that of S1 in FIG. 16A. The antenna also featured an FEP sleeve. The ablation zones shown in FIG. 19 and FIG. 20 shows the directional nature of the field emitted when operating at 8 GHz with 20 W of power for 20 s. The directional antenna was placed parallel to the surface of the tissue (FIG. 19) and energized. After application, the liver was cut to show the cross section ablation zone (FIG. 20).

A directional antenna as described above may be used for the treatment of medical conditions and/or other applications. Conditions for which the directional antenna may be used to deliver microwave energy may include, for example, percutaneous delivery into tumours that are commonly asymmetrical e.g. liver, kidney, adrenal gland;

internal surfaces of orifices that have neoplasia, dysplastic, pre-cancerous, or tumoral disease e.g. vulval intraepithelial neoplasia, oral lichen planus; or

dermatology conditions of the external surfaces of skin e.g. actinic keratosis, eczema, psoriasis

The control of the directional aspect in an application is determined by the rotation about the principal axis of the antenna. This can be facilitated by adjusting the transmission line angle directly or the assembly in which the transmission line resides.

In embodiments described above, the antenna is formed from the coaxial cable that is coupled to the microwave generator 11. In other embodiments, the antenna may comprise a separate coaxial component that is coupled to a coaxial cable that is itself coupled to the microwave generator 11. The antenna may be formed from a separate piece of coaxial cable from the coaxial cable that is attached to the microwave generator 11. The antenna may be detachable. The antenna may be disposable.

In the embodiments described above, a single directional applicator comprising a portion of a coaxial cable that is formed into a radiating element is used to radiate microwave energy into tissue.

In other embodiments, a plurality of directional applicators (for example, a plurality of the directional applicator described above) are placed around a tumour or other target. The plurality of directional applicators may direct energy specifically towards the target from a periphery of the target whilst avoiding radiating into healthy tissue.

Although certain uses for a directional antenna are described above, the directional antenna may be used for any appropriate process. In some embodiments, the directional antenna does not perform ablation. The directional antenna may perform any desired tissue heating process. For example, the directional antenna may provide more mild temperature elevation than may be used for an ablation process. The more mild temperature elevation may be used for hyperthermia. In some circumstances, lower temperatures may be used for surface applications than for penetration applications.

Whether ablation or hyperthermic treatment is performed may be dependent on energy dose. A more dense energy dose may result in heating tissue to a hotter temperature and/or heating tissue more quickly. In some circumstances, a desired result of heating may be cell death. In some circumstances, a desired result of heating may be a call heat reaction, which may not comprise cell death. Parameters (for example, parameters of the antenna and/or of the energy supplied to the antenna) may be selected in order to obtain a desired result of heating.

Embodiments of the directional antenna may be used for any appropriate process involving microwave ablation or heating (for example, hyperthermia) of human or animal tissue. The microwave ablation or heating may be performed on any human or animal subject.

In some embodiments, the antenna is introduced into the body of a patient or other subject via a catheter or trocar. In such embodiments, a diameter of the coaxial cable may be such that the antenna can fit into the catheter or trocar used. For example, different catheter sizes may be used for catheters entering different parts of the body. A diameter of the coaxial cable may be appropriate to a diameter of a body part into which the coaxial cable is to be inserted by catheter. The catheter may deliver the antenna to a position adjacent to tissue within the patient or subject, for example to the liver, heart, pancreas, or other organ. Embodiments described above may provide a compact antenna that does not extend radially beyond the radius of the coaxial cable. Such a compact antenna may easily be introduced via a catheter or trocar.

It will be understood that the present invention has been described above purely by way of example, and modifications of detail can be made within the scope of the invention. Each feature disclosed in the description, and (where appropriate) the claims and drawings may be provided independently or in any appropriate combination.

1. A microwave system comprising:
a microwave generator; and
a microwave cable apparatus comprising a coaxial cable, wherein an exposed distal portion of an inner conductor of the coaxial cable is longer than an outer conductor of the coaxial cable, and wherein at least part of the exposed distal portion of the inner conductor is bent with respect to a longitudinal axis of the coaxial cable, thereby to provide a directional radiating element;
wherein the microwave generator is configured to provide microwave energy to the cable apparatus at a frequency that provides directional radiation of microwave energy having a desired directionality from the radiating element.

2. The system according to claim 1, wherein the system is configured to perform microwave ablation of tissue and/or tissue hyperthermia.

3. The system according to claim 1, wherein the exposed distal portion is bent to form a hook shape.

4. The system according to claim 1, wherein the exposed distal portion comprises a first part that is aligned with the principal axis of the coaxial cable and a second part that is substantially parallel with, and radially offset from, the first part.

5. The system according to claim 1, wherein at least one of:
the exposed distal portion is bent with a bend angle of at least 90 degrees;
the exposed distal portion is bent such that substantially all of the exposed distal portion lies within a radius of the coaxial cable; or
the outer conductor of the coaxial cable varies in length with respect to the circumference of the coaxial cable, thereby providing a shield element.

6. (canceled)

7. (canceled)

8. The system according to claim 1, further comprising a controller configured to select the frequency of the microwave energy provided to the cable apparatus and/or a power of the microwave energy provided to the cable apparatus, wherein the frequency and/or power is selected in dependence on at least one of a reflection coefficient of the cable apparatus, a property of tissue to be treated, a volume of tissue to be treated, a type of treatment.

9. The system according to claim 1, wherein the desired directionality comprises at least one of a desired depth of penetration into tissue, a desired radiation pattern, a desired linearity, a desired profile of radiated volume.

10. The system according to claim 1, wherein the coaxial cable is flexible.

11. The system according to claim 1, further comprising a catheter or trocar into which the cable apparatus is insertable.

12. The system according to claim 1, wherein at least one of a plurality of design parameters is selected in dependence on at least one of a volume of tissue to be treated, a property of tissue to be treated, a dielectric constant of tissue to be treated, a type of treatment, the design parameters comprising at least one of:
a cable dimension;
a dimension of the radiating element;
a length of the exposed distal portion of the inner conductor;
a length of the radiating element;
a bend radius of the radiating element;
an offset distance between parts of the radiating element;
a gap between the radiating element and the outer conductor;
a size of a or the shield element;
an arc of extent of a or the shield element; or
a shape of a or the shield element.

13. The system according to claim 1, wherein the frequency is between 900 MHz and 30 GHz, optionally wherein the frequency is about 915 MHz, about 2.45 GHz, about 5.8 GHz, about 8.0 GHz, or about 24.125 GHz.

14. The system according to claim 1, wherein at least one of:
a diameter of the coaxial cable is between 0.1 mm and 25 mm; or
the radiating element is alignable with a tissue feature, so as to radiate directionally into the tissue feature.

15. (canceled)

16. A microwave cable apparatus comprising a coaxial cable, wherein an exposed distal portion of an inner conductor of the coaxial cable is longer than an outer conductor of the coaxial cable, and wherein at least part of the exposed distal portion of the inner conductor is bent with respect to a longitudinal axis of the coaxial cable, thereby to provide a directional radiating element.

17. A method of fabricating a microwave cable apparatus, the method comprising:
providing a coaxial cable, the coaxial cable comprising an inner conductor and an outer conductor;
at a distal end of the coaxial cable, selectively removing a distal portion of the outer conductor to expose a distal portion of the inner conductor; and
bending the exposed distal portion of the inner conductor to form a radiating element.

18. The method according to claim 17, wherein at least one of:
a) the exposed distal portion is bent to form a hook shape;
b) exposed distal portion comprises a first part that is aligned with the principal axis of the coaxial cable and a second part that is substantially parallel with, and radially offset from, the first part;
c) the exposed distal portion is bent with a bend angle of at least 90 degrees; or
d) the exposed distal portion is bent such that substantially all of the exposed distal portion lies within a radius of the coaxial cable.

19. The method according to claim 17, further comprising tuning a resonance and/or a distribution of microwave energy to be emitted by the microwave cable apparatus by selecting at least one of:
a cable dimension;
a dimension of the radiating element;
a length of the exposed distal portion of the inner conductor;
a length of the radiating element;
a bend radius of the radiating element;
an offset distance between parts of the radiating element; or
a gap between the radiating element and the outer conductor.

20. The method according to any of claim 17, wherein the removing of the portion of the outer conductor comprises selective removal of a portion of the outer conductor over a selected part of a circumference of the coaxial cable, thereby forming a shield element extending over a further part of the circumference of the coaxial cable, the method optionally further comprising tuning a resonance and/or a distribution of microwave energy to be emitted by the microwave cable apparatus by selecting at least one of:
a size of the shield element;
an arc of extent of the shield element; or
a shape of the shield element.

21. (canceled)

22. The method according to claim 20, further comprising shaping a distal part of the shield element to form a hood.

23. A method of performing a tissue heating process comprising:
generating microwave energy by a microwave generator;
providing the microwave energy to a microwave cable apparatus, the microwave cable apparatus comprising a coaxial cable, wherein an exposed distal portion of an inner conductor of the coaxial cable is longer than an outer conductor of the coaxial cable, and wherein at least part of the exposed distal portion of the inner conductor is bent with respect to a longitudinal axis of the coaxial cable, thereby to provide a directional radiating element; and
heating tissue by directional radiation of microwave energy having a desired directionality from the radiating element.

24. The method according to claim 23, wherein the tissue heating is so as to perform tissue ablation.

25. (canceled)
